(19) United States
(12) Patent Application Publication
(10) Pub. No.: US 2022/0008011 A1
(43) Pub. Date: Jan. 13, 2022

(54) PRINTED SENSOR COIL

(71) Applicant: St. Jude Medical, Cardiology Division, Inc., St. Paul, MN (US)

(72) Inventor: Gregory K. Olson, Elk River, MN (US)

(21) Appl. No.: 17/291,862

(22) PCT Filed: Nov. 7, 2019
(86) PCT No.: PCT/IB2019/059587

§ 371 Date: May 6, 2021

Related U.S. Application Data
(60) Provisional application No. 62/757,563, filed on Nov. 8, 2018.

(51) Int. Cl. A61B 5/00; A61B 5/287; A61B 18/14

(52) U.S. Cl. CPC A61B 5/6852; A61B 5/287; A61B 2562/0209; A61B 2562/125; A61B 18/1492

(57) ABSTRACT

A catheter can include a shaft having a proximal portion and a distal portion. The catheter can further include a tip electrode assembly with a plurality of tip electrode elements that can be mechanically combined to form a hemispherical shape. The tip electrode assembly can be affixed to the distal portion of the shaft. In some examples, the catheter can include a printed sensor coil assembly. The printed sensor coil assembly can be positioned on a concave interior surface of the shaft. In an example, the printed sensor coil assembly can include a concave coil formed by a first conductive wire segment with a concentric pattern.

### CROSS-REFERENCE TO RELATED APPLICATIONS

This application claims the benefit of U.S. application No. 62/757,563, entitled “PRINTED SENSOR COIL,” filed 8 Nov. 2018 (Attorney Docket No. CD-1459USL1/065513-001778), which is hereby incorporated by reference as though fully set forth herein.

### BACKGROUND

### a. Field

The present disclosure relates generally to an elongate medical device. In particular, the instant disclosure relates to a printed sensor coil.

### b. Background Art

Medical devices, catheters, and/or cardiovascular catheters, such as electrophysiology catheters can be used in a variety of diagnostic, therapeutic, mapping, and/or ablative procedures to diagnose and/or correct conditions such as atrial arrhythmias, including, for example, ectopic atrial tachycardia, atrial fibrillation, and atrial flutter. Arrhythmias can create a variety of conditions including irregular heart rates, loss of synchronous atrioventricular contractions and stasis of blood flow in a chamber of a heart, which can lead to a variety of symptomatic and asymptomatic ailments and even death.

A medical device can be threaded through a vasculature of a patient to a site where the diagnostic, therapeutic, mapping, and/or ablative procedure to diagnose and/or treat the condition is performed. To aid in the delivery of the medical device to the site, sensors (e.g., electrodes, electromagnetic coils) can be placed on the medical device, which can receive signals that are generated proximate to the patient from a device (e.g., electromagnetic field generator). Based on the received signals, an orientation, and/or position of the medical device can be computed.

### BRIEF SUMMARY

Various embodiments herein provide an elongate medical device. In at least one embodiment, the elongate medical device comprises a shaft, and a printed sensor coil assembly positioned on a concave interior surface of the shaft, wherein the printed sensor coil assembly comprises a concave coil formed by a first conductive wire segment with a concentric pattern.

Various embodiments herein provide a catheter. In at least one embodiment, a catheter comprises an elongated shaft, wherein the elongated shaft includes a proximal portion and a distal portion, and a tip electrode assembly comprising a plurality of tip electrode elements, where the plurality of tip electrode elements are mechanically combined to form a hemispherical shape and the tip electrode assembly is affixed to the distal portion of the elongated shaft.

### BRIEF DESCRIPTION OF THE DRAWINGS

FIG. 1 is a system diagram showing a medical device and a medical positioning system, in accordance with embodiments of the present disclosure.

FIG. 2A is a partial cross-section view of a distal end portion of a catheter including an electrode with a rectangular shape positioned on an interior side of the catheter wall, in accordance with embodiments of the present disclosure.

FIG. 2B is an isometric view of the catheter of FIG. 3A including a plurality of electrodes on the distal end portion, in accordance with embodiments of the present disclosure.

FIG. 3A is a partial cross-sectional view of a distal end portion of a catheter including a printed sensor coil with a rectangular shape positioned on an interior side of the catheter wall, in accordance with embodiments of the present disclosure.

FIG. 3B is an isometric view of the catheter of FIG. 3A including a tip electrode assembly formed from multiple elements, in accordance with embodiments of the present disclosure.

FIG. 4A is a partial cross-sectional view of a distal end portion of a catheter including a printed sensor coil with an oval shape on an interior side of the catheter wall, in accordance with embodiments of the present disclosure.

FIG. 4B is a partial cross-sectional view of a distal end portion of a catheter including a printed sensor coil with a circular shape on an interior side of the catheter wall, in accordance with embodiments of the present disclosure.

FIG. 4C is a partial cross-sectional view of a distal end portion of a catheter including a plurality of printed sensor coils with circular shapes on an interior side of the catheter wall, in accordance with embodiments of the present disclosure.

FIG. 4D is a partial cross-sectional view of a distal end portion of a catheter including a plurality of printed sensor coils with square shapes on an interior side of the catheter wall, in accordance with embodiments of the present disclosure.

FIG. 5A is a plan view of elements for tip electrode assembly, in accordance with embodiments of the present disclosure.

FIG. 5B is a plan view of a portion of a tip electrode assembly including a plurality of tip electrode elements, in accordance with embodiments of the present disclosure.

FIG. 5C is a plan view of a tip electrode assembly including a plurality of electrode elements, in accordance with embodiments of the present disclosure.

FIG. 6 is a cross-sectional view of a typical catheter shaft for an electrophysiology catheter, in accordance with embodiments of the present disclosure.

FIG. 7 is a cross-sectional view of a catheter shaft , in accordance with embodiments of the present disclosure.

FIG. 8 is a plan view of electrical contacts electrically connected to electrical conductors on a portion of a shaft, in accordance with embodiments of the present disclosure.

FIG. 9A is a plan view of an interior portion of a shaft including electrical conductors and conductive vias, in accordance with embodiments of the present disclosure.

FIG. 9B is a plan view of an exterior portion of the shaft of FIG. 9A including electrical contacts (electrically connected to the electrical conductors by the conductive vias shown in FIG. 9A), in accordance with embodiments of the present disclosure.

### DETAILED DESCRIPTION OF EMBODIMENTS

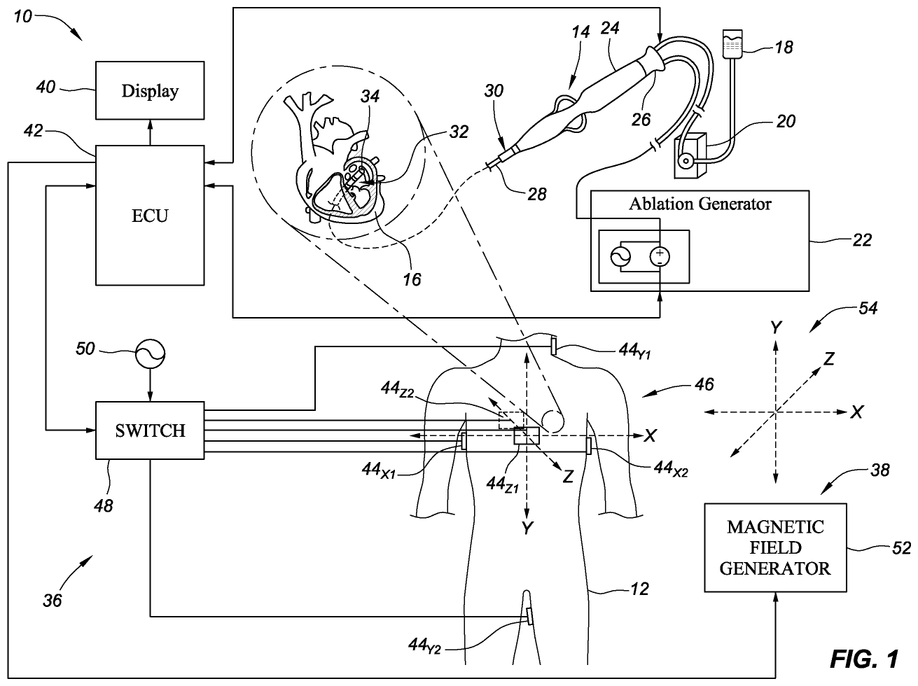

Referring now to the figures, in which like reference numerals refer to the same or similar features in the various views, FIG. 1 illustrates one embodiment of a system 10 for navigating a medical device within a body 12. In the illustrated embodiment, the medical device comprises a catheter 14 that is shown schematically entering a heart that has been exploded away from the body 12. The catheter 14, in this embodiment, is depicted as an irrigated radiofrequency (RF) ablation catheter for use in the treatment of cardiac tissue 16 in the body 12. It should be understood, however, that the system 10 may find application in connection with a wide variety of medical devices used within the body 12 for diagnosis or treatment. For example, the system 10 may be used to navigate an electrophysiological mapping catheter, an intracardiac echocardiography (ICE) catheter, or an ablation catheter using a different type of ablation energy (e.g., cryoablation, ultrasound, etc.). Further, it should be understood that the system 10 may be used to navigate medical devices used in the diagnosis or treatment of portions of the body 12 other than cardiac tissue 16. Further description of the systems and components are contained in U.S. patent application Ser. No. 13/839,963 filed on 15 Mar. 2013, which is hereby incorporated by reference in its entirety as though fully set forth herein.

Referring still to FIG. 1, the ablation catheter 14 is connected to a fluid source 18 for delivering a biocompatible irrigation fluid such as saline through a pump 20, which may comprise, for example, a fixed rate roller pump or variable volume syringe pump with a gravity feed supply from fluid source 18 as shown. The catheter 14 is also electrically connected to an ablation generator 22 for delivery of RF energy. The catheter 14 may include a handle 24; a cable connector or interface 26 at a proximal end of the handle 24; and a shaft 28 having a proximal end 30, a distal end 32, and one or more electrodes 34. The connector 26 provides mechanical, fluid, and electrical connections for conduits or cables extending from the pump 20 and the ablation generator 22. The catheter 14 may also include other conventional components not illustrated herein such as a temperature sensor, additional electrodes, and corresponding conductors or leads.

The handle 24 provides a location for the physician to hold the catheter 14 and may further provide means for steering or guiding the shaft 28 within the body 12. For example, the handle 24 may include means to change the length of one or more pull wires extending through the catheter 14 from the handle 24 to the distal end 32 of shaft 28. The construction of the handle 24 may vary.

The shaft 28 may be made from conventional materials such as polyurethane and may define one or more lumens configured to house and/or transport electrical conductors, fluids, or surgical tools. The shaft 28 may be introduced into a blood vessel or other structure within the body 12 through a conventional introducer. The shaft 28 may then be steered or guided through the body 12 to a desired location such as the tissue 16 using guide wires or pull wires or other means known in the art including remote control guidance systems. The shaft 28 may also permit transport, delivery, and/or removal of fluids (including irrigation fluids and bodily fluids), medicines, and/or surgical tools or instruments. It should be noted that any number of methods can be used to introduce the shaft 28 to areas within the body 12. This can include introducers, sheaths, guide sheaths, guide members, guide wires, or other similar devices. For ease of discussion, the term introducer will be used throughout.

The system 10 may include an electric-field-based positioning system 36, a magnetic-field-based positioning system 38, a display 40, and an electronic control unit (ECU) 42 (e.g., a processor). Each of the exemplary system components is described further below.

The electric-field-based positioning system 36 and the magnetic-field-based positioning system 38 are provided to determine the position and orientation of the catheter 14 and similar devices within the body 12. The position and orientation of the catheter 14 and similar devices within the body 12 can be determined by the system 36 and/or the system 38. The system 36 may comprise, for example, the EnSite™ NavX™ system sold by Abbott Laboratories (Abbott Park, Ill.), and described in, for example, U.S. Pat. No. 7,263,397 titled “Method and Apparatus for Catheter Navigation and Location Mapping in the Heart,” the entire disclosure of which is hereby incorporated by reference as though fully set forth herein. The systems 36 and 38 may comprise, for example, the EnSite Precision™ system sold by Abbott Laboratories (Abbott Park, Ill.). The system 36 operates based upon the principle that when low amplitude electrical signals are passed through the thorax, the body 12 acts as a voltage divider (or potentiometer or rheostat) such that the electrical potential or field strength measured at one or more electrodes 34 on the catheter 14 may be used to determine the position of the electrodes, and, therefore, of the catheter 14, relative to a pair of external patch electrodes using Ohm's law and the relative location of a reference electrode (e.g., in the coronary sinus).

In the configuration is shown in FIG. 1, the electric-field-based positioning system 36 further includes three pairs of patch electrodes 44, which are provided to generate electrical signals used in determining the position of the catheter 14 within a three-dimensional coordinate system 46. The electrodes 44 may also be used to generate EP data regarding the tissue 16. To create axes-specific electric fields within body 12, the patch electrodes are placed on opposed surfaces of the body 12 (e.g., chest and back, left and right sides of the thorax, and neck and leg) and form generally orthogonal x, y, and z axes. A reference electrode/patch (not shown) is typically placed near the stomach and provides a reference value and acts as the origin of the coordinate system 46 for the navigation system.

In accordance with this exemplary system 36 as depicted in FIG. 1, the patch electrodes include right side patch 44_{X1}, left side patch 44_{X2}, neck patch 44_{Y1}, leg patch 44_{Y2}, chest patch 44_{Z1}, and back patch 44_{Z2}; and each patch electrode is connected to a switch 48 (e.g., a multiplex switch) and a signal generator 50. The patch electrodes 44_{X1}, 44_{X2} are placed along a first (x) axis; the patch electrodes 44_{Y1}, 44_{Y2} are placed along a second (y) axis, and the patch electrodes 44_{Z1}, 44_{Z2} are placed along a third (z) axis. Sinusoidal currents are driven through each pair of patch electrodes, and voltage measurements for one or more position sensors (e.g., ring electrodes 34 or a tip electrode located near the distal end 32 of catheter shaft 28) associated with the catheter 14 are obtained. The measured voltages are a function of the distance of the position sensors from the patch electrodes. The measured voltages are compared to the potential at the reference electrode and a position of the position sensors within the coordinate system 46 of the navigation system is determined.

The magnetic-field-based positioning system 38 in this exemplary embodiment employs magnetic fields to detect the position and orientation of the catheter 14 within the body 12. The system 38 may include the GMPS system made available by MediGuide, Ltd. and generally shown and described in, for example, U.S. Pat. No. 7,386,339 titled “Medical Imaging and Navigation System,” the entire disclosure of which is hereby incorporated by reference as though fully set forth herein. In such a system, a magnetic field generator 52 may be employed having three orthogonally arranged coils (not shown) to create a magnetic field within the body 12 and to control the strength, orientation, and frequency of the field. The magnetic field generator 52 may be located above or below the patient (e.g., under a patient table) or in another appropriate location. Magnetic fields are generated by the coils and current or voltage measurements for one or more position sensors (not shown) associated with the catheter 14 are obtained. The measured currents or voltages are proportional to the distance of the sensors from the coils, thereby allowing determination of a position of the sensors within a coordinate system 54 of system 38.

The display 40 is provided to convey information to a physician to assist in diagnosis and treatment. The display 40 may comprise one or more conventional computer monitors or other display devices. The display 40 may present a graphical user interface (GUI) to the physician. The GUI may include a variety of information including, for example, an image of the geometry of the tissue 16, electrophysiology data associated with the tissue 16, graphs illustrating voltage levels over time for various electrodes 34, and images of the catheter 14 and other medical devices and related information indicative of the position of the catheter 14 and other devices relative to the tissue 16.

The ECU 42 provides a means for controlling the operation of various components of the system 10, including the catheter 14, the ablation generator 22, and magnetic generator 52 of the magnetic-field-based positioning system 38. The ECU 42 may also provide a means for determining the geometry of the tissue 16, electrophysiology characteristics of the tissue 16, and the position and orientation of the catheter 14 relative to tissue 16 and the body 12. The ECU 42 also provides a means for generating display signals used to control the display 40.

As the catheter 14 moves within the body 12, and within the electric field generated by the electric-field-based positioning system 36, the voltage readings from the electrodes 34 change, thereby indicating the location of catheter 14 within the electric field and within the coordinate system 46 established by the system 36. The ring electrodes 34 communicate position signals to ECU 42 through a conventional interface (not shown).

Additional information about magnetic sensors can be found in PCT patent application PCT/IB2018/050973, filed on 18 Feb. 2018, titled “Sensor Coil Assembly,” which is hereby incorporated by reference as if set forth fully herein.

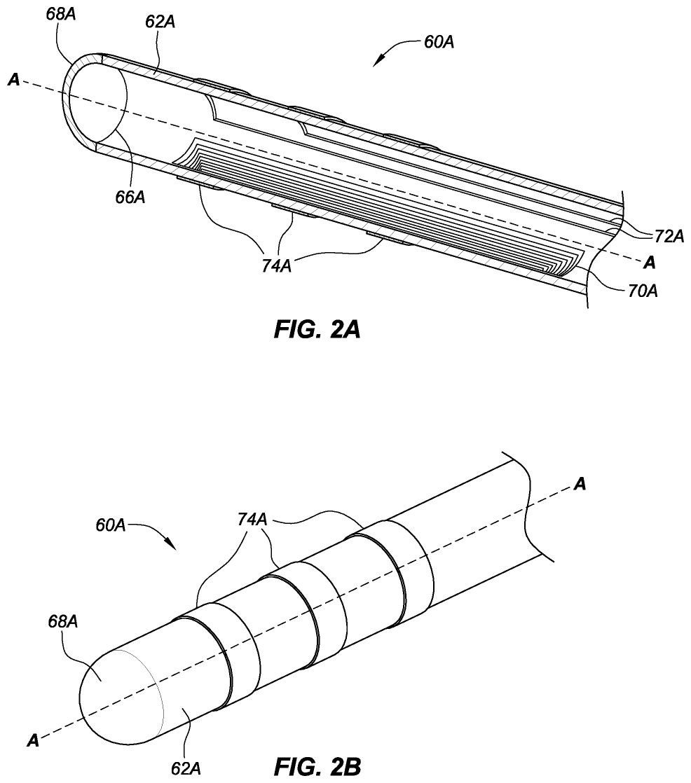

FIG. 2A is a partial cross-section view of a distal end portion of a catheter including a printed sensor coil with a rectangular shape positioned on an interior surface of the catheter wall, in accordance with embodiments of the present disclosure. A catheter 60A can include a shaft 62A, which can be elongated and can include a proximal portion 64A (not shown in FIG. 2A) and a distal portion 66A. The distal portion 66A of the shaft 62A can be affixed to, for example, a tip electrode assembly 68A. In some embodiments, a printed sensor coil 70A can be disposed in the distal portion 66A of the shaft 62A along with various electrical conductors 72A (i.e., electrical traces). The shaft 62A can have an elongated axis (represented by the line A-A) that is aligned with a central longitudinal axis of the shaft 62A. The shaft 62A can also include electrodes 74A.

In some embodiments, the printed sensor coil 70A can be shaped such that it can be appropriately positioned on an interior wall or feature (e.g., internal components including, for example, the outside diameter of a fluid lumen and/or the outside diameter of a wire management/stiffening lumen) of the shaft 62A disposed about the longitudinal axis A-A, such that the printed sensor coil 70A takes a generally concave shape (i.e., a concave sensor coil) with respect to the longitudinal axis A-A. The interior of the shaft 62A that includes the printed sensor coil 70A can include a catheter wall, a lumen, a guide wire lumen, or other interior tubular structure (e.g., insulative or tubing bundling other wires/components), or a tubular structure designed to mount a flat coil.

In some embodiments, the printed sensor coil 70A can be positioned opposite the electrical conductors 72A. In other embodiments, the printed sensor coil 70A may be placed in various other positions relative to the electrical conductors 72A. The shaft 62A can be configured to have braid in the shaft 6A (e.g., multiple layer construction) or no braid in the shaft 62A (e.g., one monolayer of printed traces on polymer).

The printed sensor coil 70A and the electrical conductors 72A can be formed using any suitable printing process. For example, such components can be formed using an ink jet printing process, an additive manufacturing process, and the like.

The printed sensor coil 70A can function as magnetic “pick-up” sensor. During use, the printed sensor coil 70A can have an operating frequency and a corresponding output signal. The operating frequency can vary depending on the design of the printed sensor coil 70A. A magnetic response of the printed sensor coil 70A is directly related to the amount of area covered by the printed sensor coil 70A. For example, a printed sensor coil will have a higher operating frequency if the size is increased (e.g., a single printed sensor coil that is longer and wider) or if the coil is stacked on one or more additional coils (e.g., two or more printed sensor coils are on top of one another, fully or partially). Additional information on printed sensor coils can be found in U.S. provisional application No. 62/460,537, titled “Printed sensor coil Assembly” filed on 17 Feb. 2017 which is hereby incorporated by reference in its entirety as though fully set forth herein.

In some embodiments, the shaft 62A can include multiple printed sensor coils 70A stacked on top of one another (not shown in FIG. 2A). The stacked coils can be the same size and/or configuration or different sizes and/or configurations (different patterns, etc.).

The physical configuration of a conductive trace can dictate characteristics of the sensor (e.g., the voltage output of the sensor). For example, the conductive trace configurations can include variations in a width of a conductive trace, a spacing between conductive traces, the overall size (i.e., sensor size of length times width) of the conductive trace, the number of layers in the conductive trace, etc. Since sensor “size” is an area (length times width), printed sensor coils could range from 0.25″ long (e.g., for 24 Fr. catheters) to 6.0″ long (for 1.0 Fr. Catheters). For example, the width of a conductive trace (i.e., trace width) in a printed sensor coil can be any suitable width from 0.0005″ to 0.010″. The spacing between the conductive traces in a printed sensor coil can range from 0.0005″ to 0.010″. In one embodiment, a single printed sensor coil 70A with a 0.002″ trace width and 0.002″ spacing that is one layer thick and 0.5″ long can produce an output of 1.21 mV. Additional layers of printed sensor coils (e.g., two printed sensor coils stacked, three sensor coils stacked, four sensor coils stacked, etc.), longer traces, or a larger trace width/height will also increase the signal output from the printed sensor coil 70A (e.g., a printed sensor coil with a smaller area, but more layers, can have the same output as a printed sensor coil with a larger area). The printed sensor coil 70A can be sized to fit within a straight section of a distal portion of the shaft 62A, which is important as this property aids in avoiding bending artifacts of the printed sensor coil 70A.

FIG. 2B is an isometric view of the catheter of FIG. 2A including a plurality of electrodes on the distal end portion, in accordance with embodiments of the present disclosure. The shaft 62A can include one or more electrodes, including the tip electrode assembly 68A and ring electrodes 74A. Although FIG. 2B illustrates three ring electrodes 74A, any other numbers of ring electrodes may be possible and are contemplated (one, two, four, five, six, etc.) The electrodes 74A are not limited to ring electrodes as any types of electrodes are suitable (e.g., surface electrodes, printed electrodes, etc.). The size of the electrodes 74A (e.g., the width relative along the longitudinal axis) and the spacing (e.g., the distance between the electrodes 74A) of the electrodes 74A can also vary. FIG. 2B shows the three ring electrodes 74A being equally spaced apart from one another. In some embodiments, the spacing between the electrodes 74A can vary, including the spacing from the tip electrode assembly 68A to the nearest ring electrode 74A.

The tip electrode assembly 68A and the ring electrodes 74A can be electrically connected to electrical conductors (e.g., the electrical conductors 72A located on an interior portion of the shaft 62A (shown in FIG. 2A)), electrically connected by conductive vias (not shown) and/or by conductors on an exterior surface of the shaft or embedded in the shaft 62A (e.g., between layers of the shaft or within a layer of the shaft).

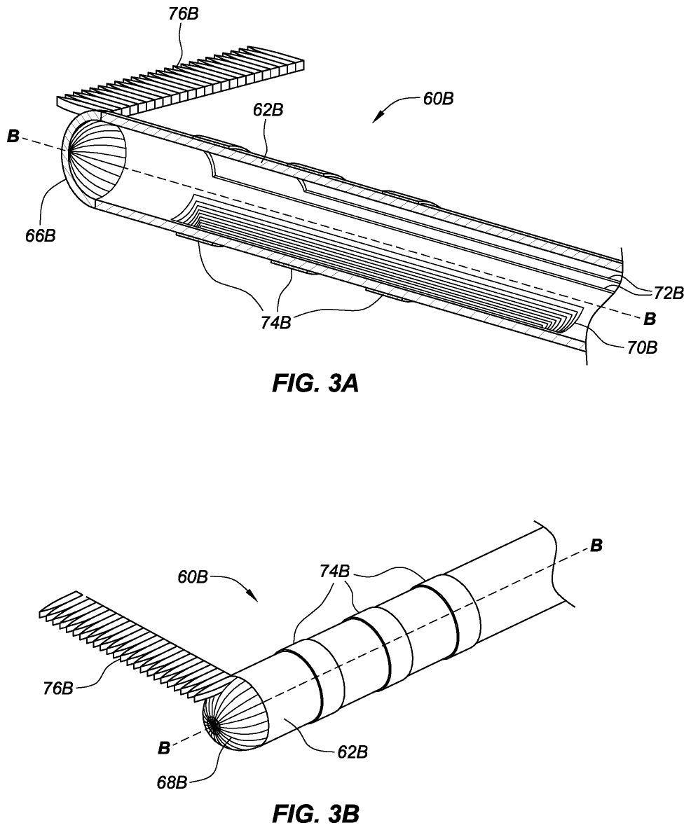

FIG. 3A is a partial cross-sectional view of a distal end portion of a catheter including a printed sensor coil with a rectangular shape positioned on an interior side of the catheter wall, in accordance with embodiments of the present disclosure. A catheter 60B can include a shaft 62B, which can be elongated and can include a proximal portion 64B (not shown in FIG. 3A) and a distal portion 66B. The distal portion 66B of the shaft 62B can be affixed to, for example, a tip electrode assembly 68B. In some embodiments, a printed sensor coil 70B can be disposed in the distal portion 66B of the shaft 62B along with various electrical conductors 72B. The shaft 62B can have an elongated axis (represented by the line B-B) that is aligned with a central longitudinal axis of the shaft 62A.

Similar to FIG. 2A, the printed sensor coil 70B can be shaped such that it may be appropriately positioned on an interior wall or feature of the shaft 62B. For example, the printed sensor coil 70B may have a generally concave shape with respect to the longitudinal axis B-B. The interior of the shaft 62B that includes the printed sensor coil 70B can include a catheter wall, a lumen, a guide wire lumen, or other interior tubular structure (e.g., insulative or tubing bundling other wires/components), or a tubular structure designed to mount a flat coil.

The tip electrode assembly 68B can be formed from a plurality of tip electrode elements 76B. The plurality of tip electrode elements 76B can fit together to form, for example, a hemispherical tip electrode assembly 68B as shown in FIG. 3A (a cross-sectional view of the hemispherical tip). In one embodiment, the plurality of tip electrode elements 76B can be wedge-shaped pieces that be mechanically combined to form a hemispherical shape.

FIG. 3B is an isometric view of the catheter of FIG. 3A including a tip electrode assembly formed from multiple elements, in accordance with embodiments of the present disclosure. The shaft 62B can include one or more electrodes, including tip electrode assembly 68B and ring electrodes 74B. FIG. 3B illustrates three ring electrodes 74B but any other number of electrodes may be present and are contemplated (e.g., one, two, four, five, six, etc.) The electrodes are not limited to ring electrodes as any types of electrodes are suitable (e.g., surface electrodes, printed electrodes, etc.). The size of the ring electrodes 74B (e.g., the width relative along the longitudinal axis) and the spacing (e.g., the distance between the ring electrodes 74B) of the electrodes can also vary. FIG. 3B shows the three ring electrodes 74B with equal spacing. In some embodiments, the spacing between the electrodes 74B can vary, including the spacing from the tip electrode assembly 68B to the nearest ring electrode 74B.

FIG. 3B shows a different view of the hemispherical tip electrode assembly 68B of FIG. 3A comprising the plurality of tip electrode elements 76B. As seen in FIGS. 3A and 3B, the plurality of tip electrode elements 76B can comprise, for example, a flat substrate (or other suitable material) and then folded/positioned to create the hemispherical tip electrode assembly 68B.

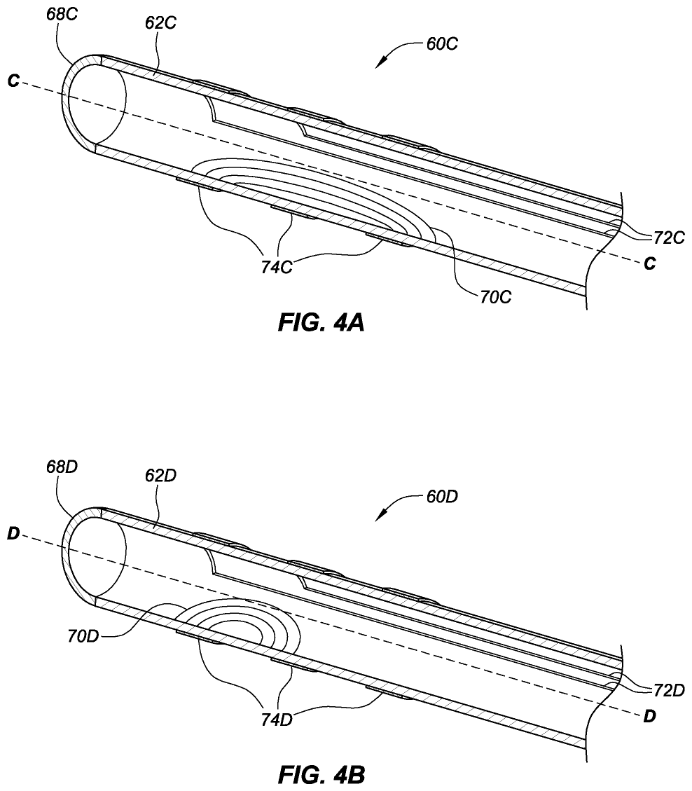

FIG. 4A is a partial cross-sectional view of a distal end portion of a catheter including a printed sensor coil with an oval shape on an interior side of the catheter wall, in accordance with embodiments of the present disclosure. A catheter 60C can include a shaft 62C, which can be elongated and can include a proximal portion 64C (not shown in FIG. 4A) and a distal portion 66C. The distal portion 66C of the shaft 62C can be affixed to, for example, a tip electrode assembly 68C. In some embodiments, a printed sensor coil 70C can be disposed in the distal portion 66C of the shaft 62C along with various electrical conductors 72C. The shaft 62C can have an elongated axis (represented by the line C-C) that is aligned with a central longitudinal axis of the shaft 62C.

In some embodiments, the oval printed sensor coil 70C can be shaped to be positioned on an interior wall or feature of the shaft 62C disposed about the elongated axis, such that the printed sensor coil 70C takes a generally concave shape with respect to the elongated axis. Portions of the interior of the shaft 62C that can be mechanically combined with the printed sensor coil 70C can include a catheter wall, a lumen, a guide wire lumen, or other interior tubular structure (e.g., insulative or tubing bundling other wires/components), or a tubular structure designed to mount a substantially flat coil.

In some embodiments, the shaft 62C can include multiple printed sensor coils 70C stacked on top of one another (not shown in FIG. 4A). The stacked coils can be the same size and/or configuration or different sizes and/or configurations (different patterns/shapes, materials, etc.).

FIG. 4B is a partial cross-sectional view of a distal end portion of a catheter including a printed sensor coil with a circular shape on an interior side of the catheter wall, in accordance with embodiments of the present disclosure. Similar to FIG. 4A, a catheter 60D can include a shaft 62D, which can be elongated and can include a proximal portion 64D (not shown in FIG. 4B) and a distal portion 66D. The distal portion 64D of the shaft 62D can be affixed to, for example, a tip electrode assembly 68D. In some embodiments, a printed sensor coil 70D can be disposed in the distal portion 66D of the shaft 62D along with various electrical conductors 72D. The shaft 62D can have an elongated axis (represented by the line D-D) that is aligned with a central longitudinal axis the shaft 62D.

In some embodiments, the circular printed sensor coil 70D can be shaped to positioned on an interior wall or feature of the shaft 62D disposed about the elongated axis, such that the printed sensor coil 70D takes a generally concave shape with respect to the elongated axis. Portions of the interior of the shaft 62D that can be mechanically combined with the printed sensor coil 70D can include a catheter wall, a lumen, a guide wire lumen, or other interior tubular structure (e.g., insulative or tubing bundling other wires/components), or a tubular structure designed to mount a substantially flat coil.

In some embodiments, the shaft 62D can include multiple printed sensor coils 70D stacked on top of one another (not shown in FIG. 2A). The stacked coils can be the same size and/or configuration or different sizes and/or configurations (different patterns, etc.).

FIG. 4C is a partial cross-sectional view of a distal end portion of a catheter including a plurality of printed sensor coils with circular shapes on an interior side of the catheter wall, in accordance with embodiments of the present disclosure. Similar to FIGS. 4A-B, a catheter 70E can include a shaft 62E, which can be elongated and can include a proximal portion 64E (not shown in FIG. 4C) and a distal portion 66E. The distal portion 66E of the shaft 62E can be affixed to, for example, a tip electrode assembly 70E. In some embodiments, a plurality of printed sensor coils 70E can be disposed in the distal portion 66E of the shaft 62E along with various electrical conductors 72E. The shaft 62E can have an elongated axis (represented by the line E-E) that is aligned with a center longitudinal axis of the shaft 62E.

As discussed herein, the plurality of circular printed sensor coils 70E can be shaped to couple with an interior wall or feature of the shaft 62E disposed about the elongated axis, such that the printed sensor coil 70E takes a generally concave shape with respect to the elongated axis. Portions of the interior of the shaft 62E that can be mechanically combined with the plurality of printed sensor coils 70E can include a catheter wall, a lumen, a guide wire lumen, or other interior tubular structure (e.g., insulative or tubing bundling other wires/components), or a tubular structure designed to mount a substantially flat coil.

FIG. 4D is a partial cross-sectional view of a distal end portion of a catheter including a plurality of printed sensor coils with square shapes on an interior side of the catheter wall, in accordance with embodiments of the present disclosure. Similar to FIGS. 4A-C, a catheter 60F can include a shaft 62F, which can be elongated and can include a proximal portion 64F (not shown in FIG. 4D) and a distal portion 66F. The distal portion 66F of the shaft 62F can be affixed to, for example, a tip electrode assembly 68F. In some embodiments, a plurality of printed sensor coils 70F can be disposed in the distal portion 66F of the shaft 62F along with various electrical conductors 72F. The shaft 62F can have an elongated axis (represented by the line F-F) that is aligned with a central longitudinal axis of the shaft 62F.

As discussed herein, the plurality of square printed sensor coils 70F can be shaped to be positioned on an interior wall or feature of the shaft 62F disposed about the elongated axis, such that the printed sensor coil 70F takes a generally concave shape with respect to the elongated axis. Portions of the interior of the shaft 62F that can be mechanically combined with the plurality of printed sensor coils 70F can include a catheter wall, a lumen, a guide wire lumen, or other interior tubular structure (e.g., insulative or tubing bundling other wires/components), or a tubular structure designed to mount a substantially flat coil.

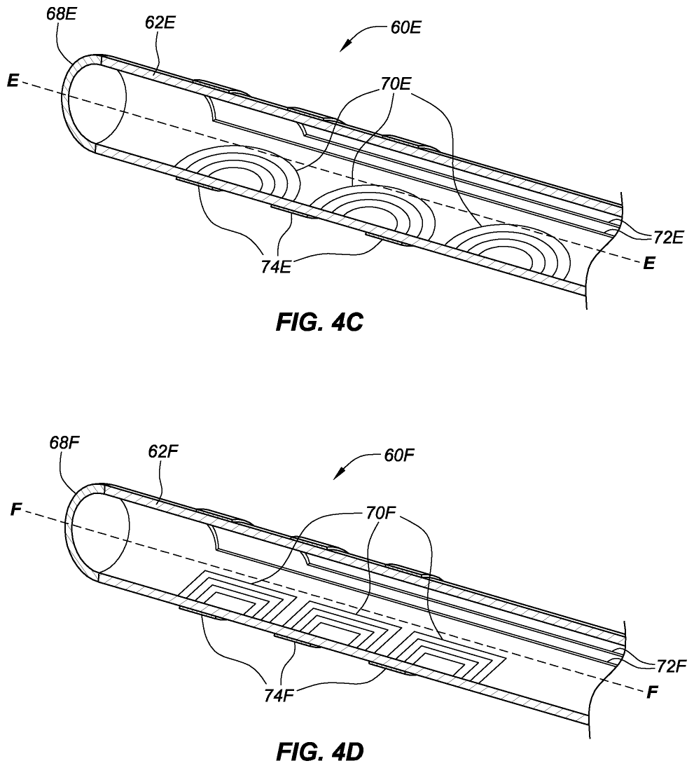

FIG. 5A is a plan view of elements for a portion of a tip electrode assembly, in accordance with embodiments of the present disclosure. A portion of a tip electrode assembly 68G can include a plurality of tip electrode elements 76G and a tip electrode element connector 78G.

The tip electrode element connector 78G can be integrated with one of the plurality of tip electrode elements 76G or it can be a separate member. The tip electrode element connector 78G can connect with a portion of each of the plurality of tip electrode elements 76G. The tip electrode element connector 78G and the tip electrode elements 76G can be connected using any suitable method (e.g., adhesive, friction fit, welding, etc.).

In one embodiment, the tip electrode elements 76G can be essentially flat elements with a distal end 80G that comes to a narrower point and a proximal end 82G that is wider than the distal end 80G. Each of the tip electrode elements 76G can include a plurality of coupling tabs 84G that allow adjacent tip electrode elements 76G to be mechanically combined to form a hemispherical shape (see FIG. 5B and related discussion) that can affix to a distal end of a catheter (e.g., the catheter 14 of FIG. 1). In addition to the plurality of coupling tabs 84G, the tip electrode element connector 78G can also allow for coupling of a portion of the tip electrode elements 76G. (See FIG. 5A and related discussion). Similar to the coupling of the tip electrode elements 76G and the tip electrode element connector 78G, the tip electrode elements 76G can be connected to each other using any suitable method (e.g., adhesive, friction fit, welding, etc.) that may include, or not include, the coupling tabs 84G.

In some embodiments (not shown), the tip electrode elements 76G can be formed from a single element (e.g., a sheet of material) and connected together (e.g., a pattern similar to a world map for a globe, etc.).

The tip electrode elements 76G can be formed from any suitable material, including common metals used for electrodes (e.g., silver, gold, platinum (and any alloys including one or more of those)) and conductive polymers (e.g., PeDOT). The tip electrode elements 76G can also include a coating (not shown) or additional layer to improve the conductivity of the tip electrode assembly 68G. Some embodiments of the tip electrode assembly 68G can include irrigation ports (not shown). Additional information about irrigation ports can be found in U.S. Pat. No. 10,016,234, titled “Flex Tip Fluid Lumen Fluid Assembly With Thermal Sensor,” filed 28 May 2015, U.S. Pat. No. 8,764,742, titled “Irrigated Catheter,” filed 4 Apr. 2007 and U.S. patent application Ser. No. 15/088,052, titled “Methods and device for delivering pulsed RF energy during catheter ablation,” filed on 31 Mar. 2016, which are hereby incorporated by reference in their entirety as though fully set forth herein.

FIG. 5B is a plan view of a portion of a tip electrode assembly including a plurality of tip electrode elements, in accordance with embodiments of the present disclosure. A portion of a tip electrode assembly 68H can include a plurality of tip electrode elements 76H (also shown in FIG. 5A). As described herein, the plurality of tip electrode elements 76H can be connected together, including a tip electrode element connector 78H, to form a hemispherical tip electrode. (Note: only half of the hemispherical tip electrode is shown in FIG. 5B).

FIG. 5C is a plan view of a tip electrode assembly including a plurality of tip electrode elements, in accordance with embodiments of the present disclosure. A tip electrode assembly 68I can include a plurality of tip electrode elements 76I, where each of the tip electrode elements is formed from a single flat element (e.g., a sheet of material). The single flat element can be configured (e.g., scored, demarked, or otherwise configured) to allow for the formation of a hemispherical shape similar to FIG. 5B (see related discussion and figure). The resulting hemispherical tip electrode assembly 68I can be affixed to a distal end of a catheter (e.g., the catheter 14 of FIG. 1). Other patterns that allow a single flat element to be formed into a hemispherical shape can also be used.

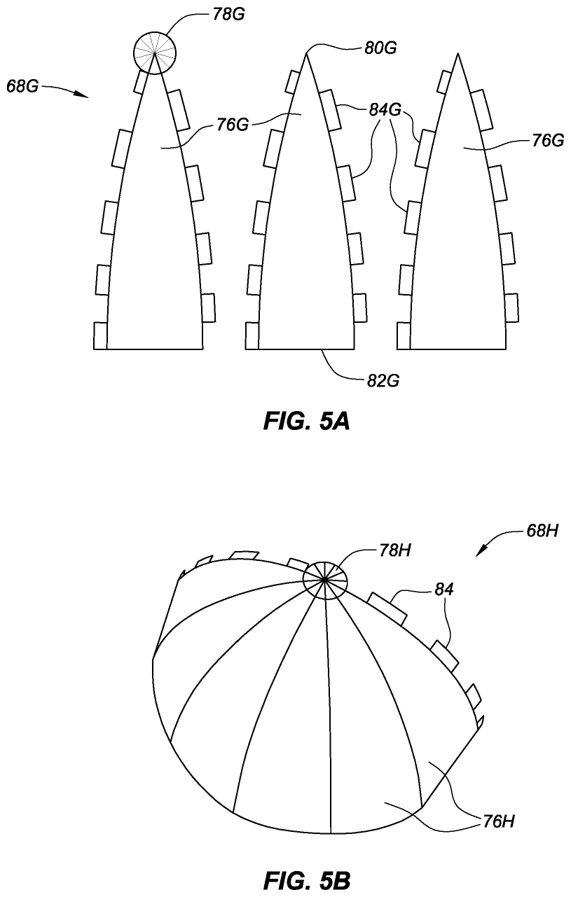

FIG. 6 is a cross-sectional view of a typical catheter shaft for an electrophysiology catheter, in accordance with embodiments of the present disclosure. A typical catheter shaft 90 can include a core 92 and an outer layer 94. The core 92 can comprise a material like a stainless steel braid. The outer layer can comprise a polymer (e.g., polyether block amide (Pebax)) or other suitable material.

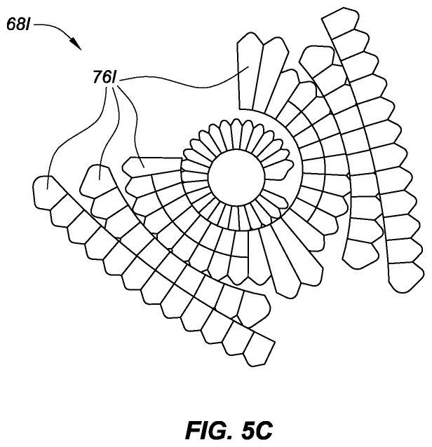

FIG. 7 is a cross-sectional view of a catheter shaft, in accordance with embodiments of the present disclosure. A catheter shaft 96 can include a core 98 (i.e., inner layer), an intermediate layer 100, and an outer layer 102. The core 98 and the intermediate layer 100 can be similar to the core 92 and the outer layer 94 of FIG. 6. However, the outer layer 102 can comprise a different material from the intermediate layer 100. For example, the outer layer 102 could comprise a liquid crystal polymer (LCP). The outer layer 102 can be less than 100 micrometers thick (e.g., 0.10 mm/0.0004″). In addition to the outer layer comprising an LCP, it can also comprise one or more electrodes/conductors (e.g., gold electrodes/conductors).

Thicknesses of the core 98, the intermediate layer 100, and the outer layer 102 can be sized to provide desired characteristics of the catheter. These catheter characteristics can comprise, for example, ability to torque, compress, bend, etc. For example, less thickness of the core 98, and/or the intermediate layer 100, and/or the outer layer 102 can allow for a catheter that is more flexible (easier to bend), more compressible, and easier to torque. Alternatively, greater thickness of the core 98, and/or the intermediate layer 100, and/or the outer layer 102 can allow for a catheter that is less flexible (i.e., harder to bend), less compressible, and harder to torque.

Size ranges for the catheter shaft 96 can range from 3 F-12 F but the configurations described herein can also be applied to catheters larger than 12 F. The outer diameter of the intermediate layer 100 could be smaller compared to typical catheters (e.g., the OD of an outer layer of the catheter 60C in FIG. 4A) to allow for the thickness of the outer layer 102 to maintain an overall OD of the catheter shaft 96 (e.g., similar to that of the catheter 60C of FIG. 4A). Further description of catheter shaft configurations with thin films are contained in U.S. patent application Ser. No. 15/265,594 filed on 14 Sep. 2016, titled “Implantable thin film devices” which is hereby incorporated by reference in its entirety as though fully set forth herein.

The catheter shaft 96 can be formed from a sheet (i.e., a rectangular substrate) that is rolled to form a tube. A surface of the sheet can comprise traces, wires, or other electrical elements (not shown in FIG. 7). Once formed into the tube, one or more electrodes can be located on a distal portion of the tube.

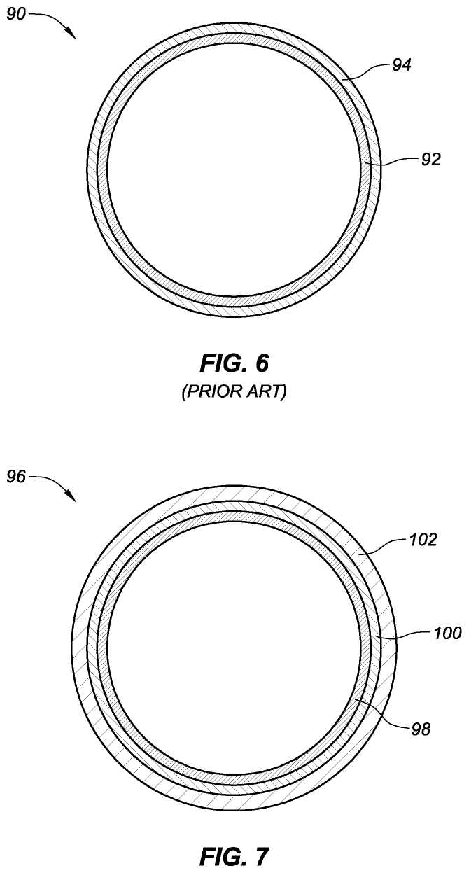

FIG. 8 is a plan view of electrical contacts electrically connected with electrical conductors on a portion of an elongate medical device shaft, in accordance with embodiments of the present disclosure. The portion of the shaft 110 can be non-planar and also non-tubular. A portion of a shaft 110 can include electrical contacts 112 that are electrically connected with electrical conductors 114 on an interior surface 116 of the shaft 110. The electrical conductors 114 can electrically connect the electrical contacts 112 with an electrical circuit (not shown in FIG. 8). For example, the electrical conductors 114 could electrically connect with a non-planar portion of a handle (e.g., handle 24 of FIG. 1) used in medical procedures, including, but not limited to, catheters used to diagnose and/or treat cardiac arrhythmias. Further description of an exemplary connection between shaft and a catheter handle are contained in U.S. provisional patent application 62/678,763 filed on 31 May 2018, titled “Catheter Handle with Compliant Circuits” which is hereby incorporated by reference in its entirety as though fully set forth herein.

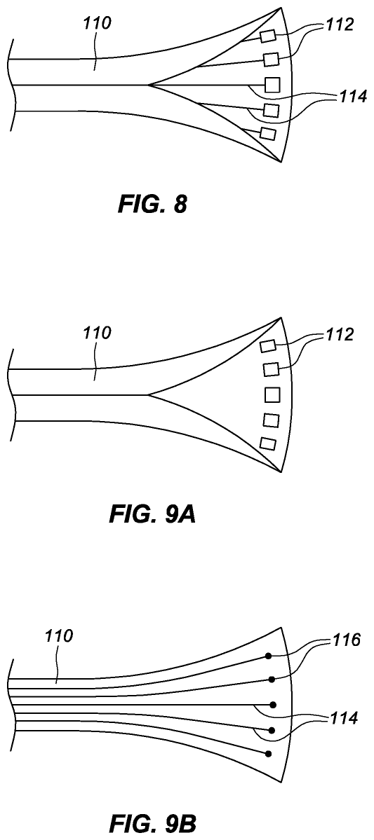

FIG. 9A is a plan view of an interior portion of a shaft including electrical conductors and conductive vias, in accordance with embodiments of the present disclosure. The portion of the shaft 110 can be non-planar and also non-tubular. An interior surface 116 of a portion of a shaft 110 can include electrical conductors that are electrically connected to conductive vias 118 (hidden from view in FIG. 9A; see FIG. 9B and related information).

FIG. 9B is a plan view of an exterior portion of the shaft of FIG. 9A including electrical contacts (electrically connected to the electrical conductors by the conductive vias shown in FIG. 9A), in accordance with embodiments of the present disclosure. The conductive vias 118 can electrically connect to the electrical contacts 112 on the exterior surface 120 of the shaft 110. Other embodiments can include the reverse configuration of the electrical conductors 114 and the electrical contacts 112 (e.g., the electrical contacts are located on the exterior surface 120 and the electrical conductors are on the interior surface 116).

Embodiments are described herein of various apparatuses, systems, and/or methods. Numerous specific details are set forth to provide a thorough understanding of the overall structure, function, manufacture, and use of the embodiments as described in the specification and depicted in the accompanying drawings. It will be understood by those skilled in the art, however, that the embodiments can be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein can be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment(s) is included in at least one embodiment. Thus, appearances of the phrases “in various embodiments,” “in some embodiments,” “in one embodiment,” or “in an embodiment,” or the like, in places throughout the specification, are not necessarily all referring to the same embodiment. Furthermore, the particular features, structures, or characteristics can be combined in any suitable manner in one or more embodiments. Thus, the particular features, structures, or characteristics illustrated or described in connection with one embodiment can be combined, in whole or in part, with the features, structures, or characteristics of one or more other embodiments without limitation given that such combination is not illogical or non-functional.

It will be appreciated that the terms “proximal” and “distal” can be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” can be used herein with respect to the illustrated embodiments. However, surgical instruments can be used in many orientations and positions, and these terms are not intended to be limiting and absolute.

Although at least one embodiment of a printed sensor coil assembly has been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the devices. Joinder references (e.g., affixed, attached, coupled, connected, and the like) are to be construed broadly and can include intermediate members between a connection of elements and relative movement between elements. As such, joinder references do not necessarily infer that two elements are directly connected and in fixed relationship to each other. It is intended that all matter contained in the above description or shown in the accompanying drawings shall be interpreted as illustrative only and not limiting. Changes in detail or structure can be made without departing from the spirit of the disclosure as defined in the appended claims.

Any patent, publication, or other disclosure material, in whole or in part, that is said to be incorporated by reference herein is incorporated herein only to the extent that the incorporated materials do not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A catheter, comprising:
a shaft including a proximal portion and a distal portion; and
a tip electrode assembly comprising a plurality of tip electrode elements, wherein the plurality of tip electrode elements are mechanically combined to form a hemispherical shape and the tip electrode assembly is affixed to the distal portion of the shaft.

2. The catheter of claim 1, wherein the tip electrode elements are mechanically combined using one or more coupling tabs of the plurality of tip electrode elements.

3. The catheter of claim 1, wherein the each of the plurality of tip electrode elements are independent of one another.

4. The catheter of claim 1, wherein each of the plurality of tip electrode elements are mechanically combined together as part of a single member.

5. The catheter of claim 1, wherein the tip electrode assembly further comprises a tip electrode element connector affixed to a distal end portion of at least one of the plurality of tip electrode elements.

6. The catheter of claim 1, wherein the tip electrode assembly further comprises one or more irrigation ports.

7. The catheter of claim 1, further comprising a printed sensor coil assembly positioned on a concave interior surface of the shaft, wherein the printed sensor coil assembly comprises a concave coil formed by a first conductive wire segment with a concentric pattern.

8. The printed sensor coil assembly of claim 7, wherein the concentric pattern comprises a circular pattern, a rectangular pattern, an oval pattern, or an elliptical pattern.

9. The printed sensor coil assembly of claim 7, wherein the first conductive wire segment comprises a conductive material deposited by at least one of a printing process and an additive manufacturing process.

10. The printed sensor coil assembly of claim 7, further comprising at least one electrode positioned on an exterior surface of the shaft and electrically connected to a conductive trace on an interior wall of the shaft.

11. The printed sensor coil assembly of claim 1, wherein the shaft comprises an inner layer, a middle layer, and, and an outer layer.

12. The printed sensor coil assembly of claim 11, wherein the inner layer comprises a braided material and the middle layer comprises a polymer.

13. The printed sensor coil assembly of claim 11, wherein a material of the outer layer of the shaft is selected from a group comprising one or more of: liquid crystal polymer, nylon, and polyether block amide.

14. The printed sensor coil assembly of claim 13, wherein the shaft further comprises a conductor positioned on a portion of the outer layer.
